(19) United States
(12) Patent Application Publication
(10) Pub. No.: US 2017/0165252 A1
(43) Pub. Date: Jun. 15, 2017

(54) PROTEIN-ENRICHED THERAPEUTIC COMPOSITION

(71) Applicant: Niconovum USA Inc., Winston-Salem, NC (US)

(72) Inventors: John-Paul Mua, Advance, NC (US); Gong Chen, Clemmons, NC (US); Thaddeus Jude Jackson, High Point, NC (US); Anthony Richard Gerardi, Winston-Salem, NC (US); Kyle Ford, Germanton, NC (US); Barry Smith Fagg, Winston-Salem, NC (US); Melissa Ann Clark, Mocksville, NC (US)

(73) Assignee: Niconovum USA Inc.

(21) Appl. No.: 14/965,069

(22) Filed: Dec. 10, 2015

(51) Int. Cl. A61K 31/465; A61K 9/20; A61K 47/26; A61K 47/36; A61K 47/10; A61K 47/46; A61K 9/00; A61K 47/48

(52) U.S. Cl. CPC A61K 31/465; A61K 9/0053; A61K 9/0056; A61K 9/2068; A61K 9/2018; A61K 47/48176; A61K 47/4823; A61K 47/36; A61K 47/10; A61K 47/46; A61K 47/26

(57) ABSTRACT

The present disclosure provides protein-enriched, nicotine-containing products, suitable for use as oral formulations. Products of the present disclosure typically include at least one nicotinic compound, at least one protein-enriched material (e.g., a tobacco-derived protein-enriched material), and at least one sugar alcohol.

### FIELD OF THE INVENTION

The present invention relates to compositions and products that contain active ingredients and, in particular, to nicotine-containing compositions and products characterized as having a pharmacological effect and that can be considered to be useful for therapeutic purposes.

### BACKGROUND OF THE INVENTION

Central nervous system (CNS) conditions, diseases, or disorders can be drug induced; can be attributed to genetic predisposition, infection or trauma; or can be of unknown etiology. They comprise neuropsychiatric disorders, neurological diseases and mental illnesses; and include neurodegenerative diseases, behavioral disorders, cognitive disorders and cognitive affective disorders. The clinical manifestations of several CNS conditions, diseases or disorders have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors).

Nicotinic compounds, such as nicotine, are capable of affecting nicotinic acetylcholinergic receptors (nAChRs). Subtypes of nAChRs exist in both the CNS and the peripheral nervous system (PNS), but the distribution of subtypes is heterogeneous. For instance, certain subtypes are predominant in vertebrate brain, others predominate at the autonomic ganglia, and others predominate at the neuromuscular junction. Activation of nAChRs by nicotinic compounds results in neurotransmitter release. See, for example, Dwoskin et al., Exp. Opin. Ther. Patents, 10: 1561-1581 (2000); Schmitt et al., Annual Reports in Med. Chem., 35: 41-51 (2000); Huang et al., J. Am. Chem. Soc., 127: 14401-14414 (2006); Arneric et al., Biochem. Pharmacol., 74: 1092-1101 (2007) and Millar, Biochem. Pharmacol., 78: 766-776 (2009), which are incorporated herein by reference.

It has been suggested that administration of nicotine, and other nicotinic compounds, can result in various pharmacological effects. See, for example, U.S. Pat. No. 5,583,140 to Bencherif et al.; U.S. Pat. No. 5,723,477 to McDonald et al.; U.S. Pat. No. 7,001,900 to Jacobsen et al.; U.S. Pat. No. 7,135,484 to Dart et al. and U.S. Pat. No. 7,214,686 to Bencherif et al.; and US Pat. Pub. Nos. 2010/0004451 to Ahmad et al. and 2011/0274628 to Borschke; which are incorporated herein by reference. As a result, it has been suggested that nicotine, and other nicotinic compounds, can exhibit utility as active ingredients in the treatment of a wide variety of conditions, diseases, and disorders, including those that affect the CNS. Additionally, administration of nicotine and nicotinic compounds has been proposed for treatment of certain other conditions, diseases, and disorders. See, for example, U.S. Pat. No. 5,604,231 to Smith et al.; U.S. Pat. No. 5,811,442 to Bencherif et al.; U.S. Pat. No. 6,238,689 to Rhodes et al. and U.S. Pat. No. 6,489,349 to Bencherif et al., which are incorporated herein by reference. Furthermore, administration of nicotine has been employed in an effort to help cigarette smokers quit smoking (i.e., as a smoking cessation aid). For example, nicotine has been an active ingredient of various types of so-called “nicotine replacement therapy” or “NRT” products. See, for example, the background art set forth in US Pat. Pub. No. 2011/0268809 Brinkley et al., which is incorporated herein by reference.

It has been proposed to administer nicotine using a transdermal patch. Representative types of nicotine-containing transdermal patch products have been marketed under the tradenames “Habitrol,” “Nicoderm,” “Nicorette,” “Nicorette CQ,” “Nicotinell” and “ProStep.” See also, for example, U.S. Pat. No. 4,597,961 to Etscom; U.S. Pat. No. 5,298,257 to Bannon et al.; U.S. Pat. No. 5,603,947 to Wong et al.; U.S. Pat. No. 5,834,011 to Rose et al.; U.S. Pat. No. 6,165,497 to Osborne et al. and U.S. Pat. No. 6,676,959 to Anderson et al., which are incorporated herein by reference. It also has been suggested that transdermal administration of nicotine can be accompanied by ingestion of other types of nicotine-containing products. See, for example, U.S. Pat. No. 5,593,684 to Baker et al.; US Pat. Pub. No. 2009/0004249 to Gonda and Fagerstrom, Health Values, 18:15 (1994), which are incorporated herein by reference.

One particularly popular way to provide for oral administration of nicotine has been through the use of nicotine-containing gum or other type of similarly chewable product. Gum forms of product generally include a gum base (e.g., typically the types of pharmaceutically acceptable gum bases available from sources such as Gum Base Co. S.p.a., Wm. J. Wrigley Jr. Company or Gumlink A/S). See, for example, the types of nicotine-containing gums, gum formulations, gum formats and configurations, gum characteristics and techniques for formulating or manufacturing gums set forth in U.S. Pat. No. 3,845,217 to Ferno et al.; U.S. Pat. No. 3,877,468 to Lichtneckert et al.; U.S. Pat. No. 3,901,248 to Lichtneckert et al.; U.S. Pat. No. 4,317,837 to Kehoe et al.; U.S. Pat. No. 4,802,498 to Ogren; U.S. Pat. No. 5,154,927 to Song et al.; U.S. Pat. No. 6,322,806 to Ream et al.; U.S. Pat. No. 6,344,222 to Cherukuri et al.; U.S. Pat. No. 6,355,265 to Ream et al.; U.S. Pat. No. 6,358,060 to Pinney et al.; U.S. Pat. No. 6,773,716 to Ream et al.; U.S. Pat. No. 6,893,654 to Pinney et al.; U.S. Pat. No. 7,101,579 Athanikar et al.; U.S. Pat. No. 7,163,705 to Johnson et al. and U.S. Pat. No. 7,208,186 to Norman et al.; US Pat. Pub. Nos. 2004/0191322 to Hansson; 2004/0194793 to Lindell et al.; 2006/0099300 to Andersen et al.; 2006/0121156 to Andersen et al.; 2006/0165842 to Andersen et al.; 2006/0204451 to Salini; 2006/0246174 to Andersen et al.; 2006/0275344 to Mody et al.; 2007/0014887 to Cherukuri et al.; 2007/0269386 to Steen et al.; 2009/0092573 to Andersen and 2010/0061940 to Axelsson et al.; which are incorporated herein by reference. Representative nicotine-containing gum products have been marketed under the tradenames “Nicorette,” “Nicotinell” and “Zonnic.”

Another way that has been employed to provide oral administration of nicotine has been through the use of nicotine-containing lozenge or tablet types of products. Nicotine-containing lozenge, mini lozenge, tablet, and microtab types of products have been marketed under the tradenames “Commit,” “Nicorette,” “Nicotinell” and “NiQuitin.” See also, for example, U.S. Pat. No. 5,110,605 to Acharya; U.S. Pat. No. 5,733,574 to Dam; U.S. Pat. No. 6,280,761 to Santus; U.S. Pat. No. 6,676,959 to Andersson et al. and U.S. Pat. No. 6,248,760 to Wilhelmsen; US Pat. Pub. Nos. 2001/0016593 to Wilhelmsen and 2010/0004294 to Axelsson et al., which are incorporated herein by reference.

A further method that has been employed to provide oral administration of nicotine has been through the use of nicotine-containing pouches or sachet types of products. See, for example, the types of pouch materials and nicotine-containing formulations set forth in U.S. Pat. No. 4,907,605 to Ray et al. and US Pat. Pub. No. 2009/0293895 to Axelsson et al., which are incorporated herein by reference. See also, for example, the types of pouch materials and pouch manufacturing techniques (e.g., pouch filling and sealing techniques) set forth in US Pat. Pub. No. 2010/0018539 to Brinkley et al., which is incorporated herein by reference. Representative nicotine-containing pouch-type products have been marketed under the tradename “Zonnic.”

Attempts have been made to incorporate nicotine into beverages (e.g., water, juices, coffee and so-called fortified beverages). See, for example, U.S. Pat. No. 6,211,194 to Westman et al.; U.S. Pat. No. 6,268,386 to Thompson; U.S. Pat. No. 6,749,882 to Fortune, Jr.; U.S. Pat. No. 7,115,297 to Stillman and U.S. Pat. No. 7,435,749 to Knight, which are incorporated herein by reference. Additionally, attempts have been made to market nicotine-containing beverages, such as certain types of beverages have been introduced commercially under the tradenames “Nic Lite,” “Nico Water,” “Nic Med,” and Nico Shot.”

Nicotine also has been administered in inhalable form, such as in the form of nasal or oral sprays. Typically, sprays are applied within the nose or mouth for absorption through nasal or oral mucosa. Various exemplary ways to administer nicotine in the form of a nasal spray are set forth in U.S. Pat. No. 4,579,858 to Ferno et al.; U.S. Pat. No. 5,656,255 to Jones and U.S. Pat. No. 6,596,740 to Jones, which are incorporated herein by reference. Various exemplary ways to administer nicotine in the form of an oral spray, such as for buccal administration, are set forth in U.S. Pat. No. 6,024,097 to Von Wielligh; US Pat. Pub. Nos. 2003/0159702 to Lindell et al.; 2007/0163610 to Lindell et al. and 2009/0023819 to Axelsson; EP 1458388 to Lindell et al. and PCT WO 2008/037470 to Axelsson et al., which are incorporated herein by reference. Various other types of inhalable formulations, and various vapor delivery devices and systems, are set forth in U.S. Pat. No. 4,284,809 to Ray; U.S. Pat. No. 4,800,903 to Ray et al.; U.S. Pat. No. 5,167,242 to Turner et al.; U.S. Pat. No. 6,098,632 to Turner et al.; U.S. Pat. No. 6,234,169 to Bulbrook et al. and U.S. Pat. No. 6,874,507 to Farr; US Pat. Pub. Nos. 2004/0034068 to Warchol et al; 2006/0018840 to Lechuga-Ballesteros; 2008/0302375 to Andersson et al. and 2009/0005423 to Gonda, which are incorporated herein by reference. Representative nicotine-containing spray-type and inhalation types of products have been marketed under the tradenames “Favor,” “Nicotrol NS,” “Quit” and “Zonnic.”

There also have been proposed numerous smoking products, flavor generators and medicinal inhalers that utilize electrical energy to vaporize or heat volatile materials (e.g., formulations that incorporate components such as tobacco-derived nicotine, glycerin, propylene glycol, organic acids and flavors), or otherwise attempt to provide the sensations of cigarette, cigar or pipe smoking without burning tobacco to a significant degree. See, for example, the various alternative smoking articles, aerosol delivery devices and heat generating sources set forth in the background art described in U.S. Pat. No. 7,726,320 to Robinson et al. and U.S. Pat. No. 8,881,737 to Collett et al., which are incorporated herein by reference. See also, for example, the various types of smoking articles, aerosol delivery devices and electrically-powered heat generating sources referenced by brand name and commercial source in U.S. Pat. Pub. No. 2015/0216232 to Bless et al., which is incorporated herein by reference. Additionally, various types of electrically powered aerosol and vapor delivery devices also have been proposed in U.S. Pat. Pub. Nos. 2014/0096781 to Sears et al. and 2014/0283859 to Minskoff et al., as well as U.S. patent application Ser. No. 14/282,768 to Sears et al., filed May 20, 2014; Ser. No. 14/286,552 to Brinkley et al., filed May 23, 2014; Ser. No. 14/327,776 to Ampolini et al., filed Jul. 10, 2014; and Ser. No. 14/465,167 to Worm et al., filed Aug. 21, 2014; all of which are incorporated herein by reference.

Various other ways to provide a source of nicotine, or to administer nicotine, have been proposed. For example, it has been suggested that nicotine can be incorporated into orally dissolving films (e.g., U.S. Pat. No. 6,709,671 to Zerbe et al.; U.S. Pat. No. 7,025,983 to Leung et al. and U.S. Pat. No. 7,491,406 to Leung et al.; and US Pat. Pub. Nos. 2006/0198873 to Chan et al.; 2006/0204559 to Bess et al. and 2010/0256197 to Lockwood et al.); oral osmotic devices (e.g., U.S. Pat. No. 5,147,654 to Place et al.); gum pads (e.g., U.S. Pat. No. 6,319,510 to Yates); oral patches (e.g., US Pat. Pub. No. 2006/0240087 to Houze et al.); lip balm (e.g., U.S. Pat. No. 7,105,173 to Rolling); dentifrice compositions and toothpicks (e.g., U.S. Pat. No. 5,176,899 to Montgomery; U.S. Pat. No. 5,035,252 to Mondre; U.S. Pat. No. 5,560,379 to Pieczenik; and US Pat. Pub. Nos. 2004/0025900 to Sampson; 2005/0058609 to Nazeri and 2006/0162732 to Winn); and other forms (e.g., U.S. Pat. No. 5,048,544 to Mascarelli; U.S. Pat. No. 6,082,368 to Brown; U.S. Pat. No. 6,319,510 to Yates and U.S. Pat. No. 6,949,264 to McGrew et al.; and US Pat. Pub. Nos. 2005/0008735 to Pearce), which are incorporated herein by reference.

It would be desirable to provide alternative compositions capable of delivering or administering nicotine via an oral route for therapeutic purposes.

### SUMMARY OF THE INVENTION

In one aspect, the present invention relates to a protein-enriched, nicotine-containing composition intended to be employed for therapeutic purposes and to methods of making such a composition. The composition is typically in a pharmaceutically acceptable form adapted for oral delivery of the composition. The composition incorporates a protein-enriched material (e.g., a protein-enriched, tobacco-derived material), at least one nicotinic compound, and at least one sugar alcohol.

In one aspect, the disclosure provides a protein-enriched pharmaceutical product comprising: a nicotinic compound; a protein-enriched, tobacco-derived material in an amount of at least about 2 percent by dry weight; and one or more sugar alcohols in an amount of at least about 10 percent by dry weight, wherein the protein-enriched, tobacco-derived material comprises at least about 60 percent tobacco-derived protein by dry weight. The nature of the tobacco-derived protein can vary. In some embodiments, the protein-enriched, tobacco-derived material comprises at least about 80 percent tobacco-derived protein by dry weight, or even higher (e.g., at least about 90 percent, at least about 95 percent, at least about 98%, at least about 99%, at least about 99.5%, or at least about 99.9% by dry weight). In certain embodiments, at least about 50 percent or at least about 80 percent of the tobacco-derived protein by dry weight is RuBisCO. In some embodiments, at least about 50 percent of the tobacco-derived protein by dry weight is F2 proteins.

In some embodiments, at least a portion of the nicotinic compound is in the form of a free base, a salt, a complex, or a solvate. For example, in certain embodiments, the nicotinic compound is nicotinic polacrilex. In some embodiments, the nicotinic compound is sorbed onto a porous particulate carrier (e.g., including, but not limited to, microcrystalline cellulose). The amount of nicotinic compound can vary and, in some embodiments, the nicotinic compound (or compounds) is present in an amount of about 0.01 to about 2 percent by dry weight.

The sugar alcohols can, in various embodiments, be selected from the group consisting of erythritol, arabitol, ribitol, isomalt, maltitol, dulcitol, iditol, mannitol, xylitol, lactitol, sorbitol, and combinations thereof. The amounts of such sugar alcohols can vary and in certain embodiments, the sugar alcohol or sugar alcohols in the disclosed pharmaceutical products are present in an amount of from about 10 percent to about 80 percent by dry weight or from about 30 percent by weight to about 70 percent by weight.

Various other components can be included in the pharmaceutical compositions and products disclosed herein. For example, in some embodiments, the compositions and products further comprise one or more binders in an amount of between about 2 percent and about 10 percent by dry weight. One exemplary binder is pregelatinized rice starch. In some embodiments, the compositions and products further comprise one or more fillers in an amount of between about 5 percent and about 50 percent by dry weight. Such fillers include, but are not limited to, fillers selected from the group consisting of maltodextrin, calcium carbonate, and combinations thereof. In certain embodiments, the compositions and products further comprise an additive selected from the group consisting of flavorants, sweeteners, binders, emulsifiers, disintegration aids, humectants, buffering agents, salts, and mixtures thereof. For example, in specific embodiments, the compositions and products can comprise glycerin and/or one or more sweeteners (e.g., including, but not limited to, sucralose).

In one specific embodiments, a protein-enriched pharmaceutical product is provided, comprising about 2 percent to about 5 percent by dry weight of the protein-enriched, tobacco-derived material; about 20 percent to about 50 percent by dry weight of the one or more sugar alcohols; about 0.01 to about 0.5 percent by dry weight of nicotine; a filler in an amount of about 30 to about 50 percent by dry weight; and a humectant in an amount of about 1 to about 10 percent by dry weight. In another specific embodiment, a protein-enriched pharmaceutical product is provided, comprising about 1 percent to about 5 percent by dry weight of the protein-enriched, tobacco-derived material; about 0.5 to about 2 percent by weight of a nicotine salt; about 50 percent to about 75 percent by dry weight of the one or more sugar alcohols; and a humectant in an amount of about 5 to about 15 percent by dry weight. In a further specific embodiment, a protein-enriched pharmaceutical product is provided, comprising about 2 percent to about 8 percent by dry weight of the protein-enriched, tobacco-derived material; about 1 to about 2 percent of a nicotine salt; about 20 percent to about 60 percent by dry weight of the one or more sugar alcohols; a filler in an amount of about 2 to about 10 percent by weight; and a binder in an amount of about 2 to about 10 percent by dry weight.

In another aspect, the present disclosure provides method of preparing a protein-enriched pharmaceutical product, comprising: combining a dry mixture comprising one or more sugar alcohols in an amount of at least about 10 percent by dry weight with a wet mixture comprising a protein-enriched, tobacco-derived material and a nicotinic compound, wherein the protein-enriched, tobacco-derived material comprises at least about 60 percent tobacco-derived protein by dry weight; and processing the combined mixture to give a protein-enriched pharmaceutical product, wherein the product comprises at least about 2 percent by dry weight of the protein-enriched, tobacco-derived material. The processing step can vary and in certain embodiments, may comprise extruding and in certain embodiments, may comprise compacting. The method can, in some embodiments, provide the protein-enriched pharmaceutical product in the form of pellets or in the form of a lozenge or tablet.

### DETAILED DESCRIPTION OF THE PREFERRED EMBODIMENTS

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water).

The present invention involves the use of nicotinic compounds for therapeutic purposes and provides compositions adapted for oral delivery of nicotinic compounds. As used herein, “nicotinic compound” refers to naturally occurring or synthetic nicotine unbound from a plant material, meaning the compound is at least partially purified and not contained within a plant structure such as a tobacco leaf. Most preferably, nicotine is naturally-occurring and obtained as an extract from a Nicotiana species (e.g., tobacco). Exemplary types of tobacco and manners of processing the tobacco are set forth in US Pat. Pub. No. 2012/0272976A1 to Byrd et al., which is incorporated herein by reference.

The nicotine can have the enantiomeric form S(−)-nicotine, R(+)-nicotine, or a mixture of S(−)-nicotine and R(+)-nicotine. Most preferably, the nicotine is in the form of S(−)-nicotine (e.g., in a form that is virtually all S(−)-nicotine) or a racemic mixture composed primarily or predominantly of S(−)-nicotine (e.g., a mixture composed of about 95 weight parts S(−)-nicotine and about 5 weight parts R(+)-nicotine). Most preferably, the nicotine is employed in virtually pure form or in an essentially pure form. Highly preferred nicotine that is employed has a purity of greater than about 95 percent, more preferably greater than about 98 percent, and most preferably greater than about 99 percent, on a weight basis. Despite the fact that nicotine can be extracted from Nicotiana species, it is highly preferred that the nicotine (and the composition and products produced in accordance with the present invention) is virtually or essentially absent of other components of tobacco (with the exception of the tobacco-derived protein-enriched material generally disclosed herein).

In embodiments wherein the nicotinic compound (e.g., nicotine) is derived from a plant of the Nicotiana species, the plant or portions thereof can be subjected to various types of processing conditions to provide the nicotine. For example, components can be separated from one another, or otherwise fractionated into chemical classes or mixtures of individual compounds. Typical separation processes can include one or more process steps (e.g., solvent extraction using polar solvents, organic solvents, or supercritical fluids), chromatography, distillation, filtration, recrystallization, and/or solvent-solvent partitioning. Exemplary extraction and separation solvents or carriers include water, alcohols (e.g., methanol or ethanol), hydrocarbons (e.g., heptane and hexane), diethyl ether methylene chloride and supercritical carbon dioxide. Exemplary techniques useful for extracting components from Nicotiana species are described in U.S. Pat. No. 4,144,895 to Fiore; U.S. Pat. No. 4,150,677 to Osborne, Jr. et al.; U.S. Pat. No. 4,267,847 to Reid; U.S. Pat. No. 4,289,147 to Wildman et al.; U.S. Pat. No. 4,351,346 to Brummer et al.; U.S. Pat. No. 4,359,059 to Brummer et al.; U.S. Pat. No. 4,506,682 to Muller; U.S. Pat. No. 4,589,428 to Keritsis; U.S. Pat. No. 4,605,016 to Soga et al.; U.S. Pat. No. 4,716,911 to Poulose et al.; U.S. Pat. No. 4,727,889 to Niven, Jr. et al.; U.S. Pat. No. 4,887,618 to Bernasek et al.; U.S. Pat. No. 4,941,484 to Clapp et al.; U.S. Pat. No. 4,967,771 to Fagg et al.; U.S. Pat. No. 4,986,286 to Roberts et al.; U.S. Pat. No. 5,005,593 to Fagg et al.; U.S. Pat. No. 5,018,540 to Grubbs et al.; U.S. Pat. No. 5,060,669 to White et al.; U.S. Pat. No. 5,065,775 to Fagg; U.S. Pat. No. 5,074,319 to White et al.; U.S. Pat. No. 5,099,862 to White et al.; U.S. Pat. No. 5,121,757 to White et al.; U.S. Pat. No. 5,131,414 to Fagg; U.S. Pat. No. 5,131,415 to Munoz et al.; U.S. Pat. No. 5,148,819 to Fagg; U.S. Pat. No. 5,197,494 to Kramer; U.S. Pat. No. 5,230,354 to Smith et al.; U.S. Pat. No. 5,234,008 to Fagg; U.S. Pat. No. 5,243,999 to Smith; U.S. Pat. No. 5,301,694 to Raymond et al.; U.S. Pat. No. 5,318,050 to Gonzalez-Parra et al.; U.S. Pat. No. 5,343,879 to Teague; U.S. Pat. No. 5,360,022 to Newton; U.S. Pat. No. 5,435,325 to Clapp et al.; U.S. Pat. No. 5,445,169 to Brinkley et al.; U.S. Pat. No. 6,131,584 to Lauterbach; U.S. Pat. No. 6,298,859 to Kierulff et al.; U.S. Pat. No. 6,772,767 to Mua et al.; and U.S. Pat. No. 7,337,782 to Thompson and US Pat. Appl. Pub. No. 2013/0078307 to Holton, all of which are incorporated herein by reference. See also, the types of separation techniques set forth in Brandt et al., LC-GC Europe, p. 2-5 (March, 2002) and Wellings, A Practical Handbook of Preparative HPLC (2006), which are incorporated herein by reference. In addition, the plant or portions thereof can be subjected to the types of treatments set forth in Ishikawa et al., Chem. Pharm. Bull., 50, 501-507 (2002); Tienpont et al., Anal. Bioanal. Chem., 373, 46-55 (2002); Ochiai, Gerstel Solutions Worldwide, 6, 17-19 (2006); Coleman, III, et al., J. Sci. Food and Agric., 84, 1223-1228 (2004); Coleman, III et al., J. Sci. Food and Agric., 85, 2645-2654 (2005); Pawliszyn, ed., Applications of Solid Phase Microextraction, RSC Chromatography Monographs, (Royal Society of Chemistry, UK) (1999); Sahraoui et al., J. Chrom., 1210, 229-233 (2008); and U.S. Pat. No. 5,301,694 to Raymond et al., which are all incorporated herein by reference.

Nicotinic compounds of the invention can include nicotine in free base form, salt form, as a complex, or as a solvate. See, for example, the discussion of nicotine in free base form in US Pat. Pub. No. 2004/0191322 to Hansson, which is incorporated herein by reference. At least a portion of the nicotinic compound can be employed in the form of a resin complex of nicotine where nicotine is bound in an ion exchange resin such as nicotine polacrilex. See, for example, U.S. Pat. No. 3,901,248 to Lichtneckert et al.; which is incorporated herein by reference. At least a portion of the nicotine can be employed in the form of a salt. Salts of nicotine can be provided using the types of ingredients and techniques set forth in U.S. Pat. No. 2,033,909 to Cox et al., Perfetti, Beitrage Tabakforschung Int., 12, 43-54 (1983), and U.S. patent application Ser. No. 14/721,283 to Dull, filed May 26, 2015. Additionally, salts of nicotine have been available from sources such as Pfaltz and Bauer, Inc. and K&K Laboratories, Division of ICN Biochemicals, Inc. Exemplary pharmaceutically acceptable nicotine salts include nicotine salts of tartrate (e.g., nicotine tartrate and nicotine bitartrate) chloride (e.g., nicotine hydrochloride and nicotine dihydrochloride), sulfate, perchlorate, ascorbate, fumarate, citrate, malate, lactate, aspartate, salicylate, tosylate, succinate, pyruvate, and the like; nicotine salt hydrates (e.g., nicotine zinc chloride monohydrate), and the like. In certain embodiments, at least a portion of the nicotinic compound is in the form of a salt with an organic acid moiety, including, but not limited to, levulinic acid as discussed in U.S. patent application Ser. No. 12/769,335 and International Application No. PCT/US2011/033928, both to Brinkley et al., which are incorporated herein by reference.

In one embodiment, the nicotinic compound is sorbed onto a porous particulate carrier material, such as microcrystalline cellulose (MCC) prior to incorporation within the compositions of the invention. In one embodiment, the MCC materials used in the invention have an average particle size range of about 15 to about 250 microns. Exemplary MCC materials include various grades of AVICEL® and VIVACEL® materials. See, for example, US Pat. Pub. No. 2004/0191322 to Hansson, which is incorporated by reference herein. In certain embodiments, multiple forms of nicotinic compounds could be sorbed onto the particulate carrier, including any of the various nicotinic compound combinations discussed herein. In some embodiments, the nicotinic compound and, optionally, an organic acid moiety can be sorbed onto the particulate carrier by, for example, dissolving the nicotinic compound (and, optionally, an organic acid moiety) in a hydrophilic solvent (such as water, alcohol, or mixtures thereof) and combining the solution with the particulate carrier, followed by drying to remove the solvent. The particulate carrier material with the sorbed nicotine and, optionally, organic acid moiety, can be combined with other carriers or excipients in order to provide a composition adapted for oral delivery of the active ingredient.

The protein-enriched material in the compositions and products disclosed herein is a material comprising at least about 50% protein by dry weight, at least about 60% protein by dry weight, at least about 70% protein by dry weight, at least about 80% protein by dry weight, at least about 90% protein by dry weight, at least about 95% protein by dry weight, at least about 98% protein by dry weight, or at least about 99% protein by dry weight. The protein-enriched material is generally plant-derived protein-enriched material. It is understood that the water-soluble portion of plant biomass generally consists of two fractions. One fraction predominantly comprises ribulose-1,5-bisphosphate carboxylase oxygenase (commonly referred to as RuBisCO), whose subunit molecular weight is about 550 kD (commonly referred to as a “Fraction 1 protein” or “F1 protein”). RuBisCO may comprise up to about 25% of the total protein content of a leaf and up to about 10% of the solid matter of a leaf. A second fraction (“Fraction 2 protein” or “F2 protein”) generally contains a mixture of proteins and peptides with molecular weights ranging from about 3 kD to about 100 kD and may also contain other compounds including sugars, vitamins, alkaloids, flavors, and amino acids. The protein-enriched material incorporated within the compositions and products of the present disclosure can comprise Fraction 1 protein and/or Fraction 2 protein.

In some embodiments, the protein-enriched material is a RuBisCO-enriched material, e.g., a material comprising at least about 50% RuBisCO by dry weight, at least about 60% RuBisCO by dry weight, at least about 70% RuBisCO by dry weight, at least about 80% RuBisCO by dry weight, at least about 90% RuBisCO by dry weight, at least about 95% RuBisCO by dry weight, at least about 98% RuBisCO by dry weight, or at least about 99% RuBisCO by dry weight. In some embodiments, the protein-enriched material is an F2 protein-enriched material, e.g., a material comprising at least about 10% F2 protein by dry weight, at least about 20% F2 protein by dry weight, at least about 30% protein by dry weight, at least about 50% F2 protein by dry weight, at least about 60% F2 protein by dry weight, at least about 70% F2 protein by dry weight, at least about 80% F2 protein by dry weight, at least about 90% F2 protein by dry weight, at least about 95% F2 protein by dry weight, at least about 98% F2 protein by dry weight, or at least about 99% F2 protein by dry weight.

Where a combination of RuBisCO and F2 protein is used, the predominant protein can be either RuBisCO or F2 protein. In some embodiments, the protein in the protein-enriched material as a whole comprises at least about 50% RuBisCO by dry weight, at least about 60% RuBisCO by dry weight, at least about 70% RuBisCO by dry weight, at least about 80% RuBisCO by dry weight, at least about 90% RuBisCO by dry weight, at least about 95% RuBisCO by dry weight, at least about 98% RuBisCO by dry weight, or at least about 99% RuBisCO by dry weight. In some embodiments, the protein in the protein-enriched material as a whole comprises at least about 50% F2 protein by dry weight, at least about 60% F2 protein by dry weight, at least about 70% F2 protein by dry weight, at least about 80% F2 protein by dry weight, at least about 90% F2 protein by dry weight, at least about 95% F2 protein by dry weight, at least about 98% F2 protein by dry weight, or at least about 99% F2 protein by dry weight.

Particularly preferred protein-enriched materials for use in the compositions and products disclosed herein comprise RuBisCO. RuBisCO has been found to exhibit good nutritional properties and is colorless, tasteless, and odorless. Further, certain physical properties of RuBisCO render it advantageous for use in such products, as it has excellent binding, gelling, solubility, and emulsifying behavior. RuBisCO and F2 protein can be extracted from a wide array of plant materials and exemplary methods are described, for example, in U.S. Pat. No. 4,268,632 to Wildman et al., U.S. Pat. No. 4,340,676 to Bourke; U.S. Pat. No. 4,400,471 to Johal; U.S. Pat. No. 4,588,691 to Johal; and U.S. Pat. No. 6,033,895 to Garger et al., which are incorporated herein by reference. In certain preferred embodiments, the protein-enriched material is a tobacco-derived protein-enriched material. One exemplary tobacco-derived protein-enriched material is described in US Pat. App. Publ. No. 2014/0271952 to Mua et al., which is incorporated herein by reference in its entirety. Further details regarding additional processing of such materials to increase the purity thereof are provided in US Pat. App. Publ. No. 2014/0343254 to Gerardi et al., which is incorporated herein by reference. Where the protein-enriched material is tobacco-derived, it is preferred that the protein-enriched material is substantially pure so as to avoid the incorporation of significant amounts of, e.g., tobacco, processed tobacco components, and the types of components of tobacco traditionally present within tobacco-containing cigarettes, cigars, pipes, or smokeless forms of tobacco products.

The form of the protein-enriched material (i.e., RuBisCO-enriched material, combined RuBisCO/F2 protein-enriched material, and/or F2 protein-enriched material) used within the disclosed compositions and according to the methods of the present disclosure can vary. Typically, these materials are in solid, liquid, or semi-solid or gel forms and formulations comprising such materials can be used in concrete, absolute, or neat form. Solid forms of the protein-enriched materials described herein can include spray-dried and freeze-dried forms. Liquid forms of the protein-enriched materials described herein can include formulations contained within aqueous or organic solvent carriers.

The amount of protein-enriched material incorporated within a pharmaceutical composition or product according to the present disclosure can depend on the desired function of the protein-enriched material, the chemical makeup of the protein-enriched material, and the type of pharmaceutical composition to which the protein-enriched material is added. The amount of protein-enriched material added to a given composition can vary, but will typically not exceed about 50 weight percent based on the total dry weight of the composition to which the protein-enriched material is added. For example, the amount of protein-enriched material added to a pharmaceutical composition as disclosed herein may be in the range of about 0.25 to about 30 weight percent, about 10 to about 30 weight percent, or about 1 to about 10 weight percent, based on the total dry weight of the pharmaceutical composition.

The protein-enriched material can serve various functions within the pharmaceutical compositions and products disclosed herein. For example, in some embodiments, the protein-enriched material can function as a gelling and/or binding agent. In some embodiments, the protein-enriched material (e.g., RuBisCO-enriched material) can serve as a replacement for at least a portion of the hydrocolloids (including, but not limited to, starch, gelatin, pectin, gums, and the like) in various products. In some embodiments, the protein-enriched material (e.g., RuBisCO-enriched material) can serve as a replacement for at least a portion of the fillers.

The compositions of the invention possess a form that is pharmaceutically effective and pharmaceutically acceptable. That is, the composition most preferably does not incorporate to any appreciable degree, or does not purposefully incorporate, components of tobacco, other than the components described hereinabove (i.e., nicotine and/or tobacco-derived protein-enriched material). As such, pharmaceutically effective and pharmaceutically acceptable compositions do not include any additional material that can be characterized as tobacco, processed tobacco components, or components of tobacco traditionally present within tobacco-containing cigarettes, cigars, pipes, or smokeless forms of tobacco products. Highly preferred compositions include less than 0.5 weight percent of tobacco components other than nicotine and the protein-enriched material, more often less than about 0.25 weight percent, and typically are entirely absent or devoid of components of tobacco, processed tobacco components, or components derived from tobacco, other than nicotine and the protein-enriched material disclosed herein.

The pharmaceutical compositions of the invention may be conveniently made available in a unit dosage form, whereby such formulations may be prepared by any of the methods generally known in the pharmaceutical arts. Generally speaking, such methods of preparation comprise combining (by various methods) an active agent with a suitable carrier or other adjuvant, which may consist of one or more ingredients (including the protein-enriched material disclosed herein). The combination of the active ingredient with the one or more adjuvants is then physically treated to present the formulation in a suitable form for delivery (e.g., shaping into a tablet or forming an aqueous suspension).

The nicotine-containing pharmaceutical compositions of the invention can incorporate various pharmaceutically acceptable excipients. By “pharmaceutically acceptable carrier” or “pharmaceutically acceptable excipient” is intended a carrier or excipient that is conventionally used in the art to facilitate the storage, administration, and/or the healing effect of an active agent (e.g., a nicotinic compound). The carrier(s) must be pharmaceutically acceptable in the sense of being compatible with the other ingredients of the formulation and not unduly deleterious to the recipient thereof. A carrier may also reduce any undesirable side effects of the agent. See, Wang et al. (1980) J. Parent. Drug Assn. 34(6):452-462, herein incorporated by reference in its entirety. Other exemplary pharmaceutical excipients and/or additives suitable for use in the compositions according to the invention are listed in Remington: The Science & Practice of Pharmacy, 21^{st} ed., Lippincott Williams & Wilkins (2006); in the Physician's Desk Reference, 64^{th} ed., Thomson PDR (2010); and in Handbook of Pharmaceutical Excipients, 6^{th} ed., Eds. Raymond C. Rowe et al., Pharmaceutical Press (2009), which are incorporated herein by reference.

The various excipients can vary, and the selection and amount of each excipient can depend upon factors such as the ultimate form and function of product that is desired. See, for example, the types of ingredients, relative amounts and combinations of ingredients, nicotine-containing formulations and preparation processes for nicotine-containing products set forth in U.S. Pat. No. 5,512,306 to Carlsson et al.; U.S. Pat. No. 5,525,351 to Dam; U.S. Pat. No. 5,549,906 to Santus; U.S. Pat. No. 5,711,961 to Reiner et al.; U.S. Pat. No. 5,811,126 to Krishnamurthy; U.S. Pat. No. 5,939,100 to Albrechtsen et al.; U.S. Pat. No. 6,024,981 to Khankari et al.; U.S. Pat. No. 6,083,531 to Humbert-Droz et al.; U.S. Pat. No. 6,090,401 to Gowan, Jr. et al.; U.S. Pat. No. 6,110,495 to Dam; U.S. Pat. No. 6,248,760 to Wilhelmsen; U.S. Pat. No. 6,280,761 to Santus; U.S. Pat. No. 6,426,090 to Ream et al.; U.S. Pat. No. 6,569,463 to Patel et al.; U.S. Pat. No. 6,583,160 to Smith et al.; U.S. Pat. No. 6,585,997 to Moro et al.; U.S. Pat. No. 6,676,959 to Andersson et al.; U.S. Pat. No. 6,893,654 to Pinney et al.; U.S. Pat. No. 7,025,983 to Leung et al.; and U.S. Pat. No. 7,163,705 Johnson et al.; US Pat. Pub. Nos. 2003/0176467 to Andersson et al.; 2003/0235617 to Martino et al.; 2004/0096501 to Vaya et al.; 2004/0101543 to Liu et al.; 2004/0191322 to Hansson; 2005/0053665 to Ek et al.; 2005/0123502 to Chan et al.; 2008/0038209 to Andersen et al.; 2008/0286341 to Andersson et al.; 2009/0023819 to Axelsson; 2009/0092573 to Andersen; 2010/0004294 to Axelsson et al.; and 2010/0061940 to Axelsson et al.; which are incorporated herein by reference.

In addition to the protein-enriched material and the nicotinic compound(s), the pharmaceutical compositions of the present disclosure typically include at least one sugar or sugar alcohol. Although sucrose can be used in the preparation of the nicotine-containing products of the present invention, the products are typically sugar-free products, comprising one or more sugar substitutes. “Sugar-free” as used herein is intended to include products having less than about 1/15th sugar by weight, or less than about 1/10th sugar by weight. The sugar substitute can be any sugarless material (i.e., sucrose-free material) and can be natural or synthetically produced. The sugar substitute used in the invention can be nutritive or non-nutritive. For example, the sugar substitute is commonly a sugar alcohol. Sugar alcohols are polyols derived from monosaccharides or disaccharides that have a partially or fully hydrogenated form. Exemplary sugar alcohols have between about 4 and about 20 carbon atoms and include erythritol, arabitol, ribitol, isomalt, polyglycitol, maltitol, dulcitol, iditol, mannitol, xylitol, lactitol, sorbitol, and combinations thereof (e.g., hydrogenated starch hydrolysates). Sugar alcohols can fulfill multiple functions, such as providing sweetness, enhancing certain organoleptic properties such as texture and mouthfeel, enhancing cohesiveness or compressibility of the product, and the like.

A combination of sugar alcohols is typically utilized in the present invention. The exact combination of sugar alcohols used in any given formulation can be selected based on a number of factors, including laxation threshold, relative sweetness, calorie content, glycemic index, degree of hygroscopicity, and the like. In one embodiment, a combination of two or more of xylitol, maltitol, and sorbitol is used. Interestingly, this particular combination of sugar alcohols provides a chewy product that has a moderate cooling effect in the mouth. In certain embodiments, a combination of sorbitol, erythritol, and isomalt or maltitol, erythritol, and isomalt is used in a “chewy gel” type product. In certain embodiments, sucrose and sorbitol are used in a “pastille” type product. In some embodiments, xylitol, sorbitol, and maltitol are used in an “extruded rod” type product. In some embodiments, mannitol or a combination of isomalt and erythritol is used in a “pellet” type product.

Where a combination of sugar alcohols is used, the ratio of the sugar alcohols with respect to one another can vary. In some embodiments, each sugar alcohol is provided in roughly the same weight percentage. In other embodiments, the sugar alcohols can be provided in different weight percentages (with one or more sugar alcohols being principal sugar alcohol component(s) and one or more sugar alcohols being minor sugar alcohol component(s)).

The total sugar alcohol content of the compositions of the invention will typically range from about 5 to about 75 weight percent based on total dry weight of the product, such as about 10 to about 50 weight percent, or about 10 to about 25 weight percent, about 20 to about 50 weight percent, or about 20 to about 75 weight percent. The total sugar alcohol content of the product will typically be at least about 10 weight percent, or at least about 15 weight percent based on total dry weight of the product. The sugar alcohol content of the products will typically not exceed about 90 weight percent, such as no more than about 85 weight percent, no more than about 80 weight percent, no more than 75 weight percent, or no more than about 50 weight percent.

Other pharmaceutically acceptable components may be added to the products of the invention. For example, in certain embodiments, the nicotine-containing pharmaceutical composition further comprises a salt. The presence of a salt in the composition may act to suppress bitterness and/or enhance sweetness. Any type of salt can be used. Common table salt (sodium chloride, NaCl) is typically used according to the present invention, but other types of salts are intended to be encompassed as well. The amount of salt added may vary, but typically ranges from 0% to about 10%, for example from about 1% to about 8% or from about 2% to about 6% by weight of the pharmaceutical composition mixture. In some embodiments, a somewhat salty taste is a desirable feature of the pharmaceutical composition.

In some embodiments, the composition according to the invention further comprises one or more buffering agents and/or pH adjusters (e.g., acids or bases). Certain exemplary buffering agents and/or pH adjusters include, but are not limited to, magnesium oxide, magnesium hydroxide, potassium carbonate, sodium carbonate, potassium bicarbonate, sodium bicarbonate, citric acid, or mixtures thereof. In some embodiments, one or more buffering agents and/or pH adjusters are added to the mixture to ensure that the final pharmaceutical composition has a pH within a desirable range. Exemplary pH ranges in such compositions are generally from about 6-11, and often about 7-10 (e.g., about 7 or about 8). In such embodiments, the amount of buffering agent and/or pH adjuster added to the composition mixture is simply that amount required to bring the formulation to, or keep the formulation at, the desired pH. The amount of buffering agent and/or pH adjuster added to any given formulation can be readily calculated by one skilled in the art and may comprise, for example, about 0.5% to about 1% by weight of the mixture. It is noted that in certain embodiments, a basic pH is not necessary in the products of the present invention. Accordingly, certain products of the present invention have a pH of less than about 6 or less than about 5 (e.g., from about 4 to about 6).

Various food-grade buffering agents are known and can be used to adjust the pH of the products of the present invention. Suitable buffering agents include those selected from the group consisting of acetates, glycinates, phosphates, glycerophosphates, citrates such as citrates of alkaline metals, carbonates, hydrogen carbonates, and borates, and mixtures thereof. In certain embodiments, the buffering agent is an amino acid, as taught for example, in US Pat. Pub. No. 2008/0286341 to Andersson et al. and PCT Appl. No. WO2008/040371 to Andersson et al., which are both incorporated herein by reference. As noted therein, various amino acids and salts thereof are useful for this purpose, including, but not limited to, arginine, asparagine, glutamic acid, glutamine, glycine, histidine, isoleucine, leucine, lysine, methionine, phenylalanine, serine, threonine, valine, cysteic acid, N-glycylglycine, and ornithine. In certain embodiments, N-glycylglycine or L-lysine is added as a buffering agent. In some embodiments, an amino acid buffering agent is used in combination with another amino acid buffering agent and/or in combination with one or more non-amino acid buffering agents. In certain embodiments, the optional pH adjusting agent is a base (e.g., NaOH). In certain embodiments, L-lysine and NaOH are added to the compositions of the present invention.

In some embodiments, one or more additional sweeteners are added to the compositions of the present invention. The one or more additional sweeteners can comprise any natural or artificial sweetener, including, but not limited to, sugar or any of the sugar substitutes described previously. In certain embodiments, the sweetener can include, glycyrrhizin, glycerol, inulin, lactitol, lactose, mabinlin, maltitol, mannitol, miraculin, monatin, monellin, osladin, pentadin, polydextrose, sorbitol, stevia, tagatose, thaumatin, acesulfame potassium, alitame, aspartame, cyclamate, dulcin, glucin, neotame, saccharin, sorbitol, sucralose, xylitol, and combinations thereof. In certain embodiments, the sweetener comprises sucralose (1,6-Dichloro-1,6-dideoxy-β-D-fructofuranosyl-4-chloro-4-deoxy-α-D-galactopyranoside). The amount of sweetener added can vary, but is typically that amount required for a sufficiently “sweet” taste. For example, sweetener can be added to make the sweetness of the nicotine-containing pharmaceutical composition comparable to that of sugar. In particular embodiments, sucralose is added in an amount of about 0.5% to about 2% by weight of the product mixture, often in an amount of about 1% by weight of the mixture.

Various natural and/or artificial flavorants can also be added to the pharmaceutical compositions of the present invention. As used herein, a “flavorant” or “flavoring agent” is any flavorful or aromatic substance capable of altering the sensory characteristics associated with the pharmaceutical composition. Exemplary sensory characteristics that can be modified by the flavorant include, taste, mouthfeel, moistness, coolness/heat, and/or fragrance/aroma. The flavorants can be natural or synthetic, and the character of these flavors can be described as, without limitation, fresh, sweet, herbal, confectionery, floral, fruity or spicy. Specific types of flavors include, but are not limited to, vanilla (e.g., vanillin optionally in complexed form), coffee, chocolate, cream, mint, spearmint, menthol, peppermint, wintergreen, lavender, cardamon, nutmeg, cinnamon, clove, cascarilla, sandalwood, honey, jasmine, ginger, anise, sage, licorice, and fruit flavors such as lemon, orange, apple, peach, lime, cherry, and strawberry. See also, Leffingwill et al., Tobacco Flavoring for Smoking Products, R. J. Reynolds Tobacco Company (1972), which is incorporated herein by reference. Flavorings also can include components that are considered moistening, cooling or smoothening agents, such as eucalyptus. Flavorings can also include sensates, which can add a range of tactile, organoleptic properties to the pharmaceutical compositions. For example, sensates can provide a warming, cooling, or tingling sensation. These flavors may be provided neat (i.e., alone) or in a composite (e.g., spearmint and menthol, or orange and cinnamon). Flavorants of this type can be present in an amount of from about 0.5% to about 15%, often between about 1% and about 5% by weight of the composition. In certain embodiments, the flavorant is present in any amount of at least about 0.5% by weight, at least about 0.75% by weight of the composition, at least about 1% by weight of the composition, or at least about 2% by weigh of the composition.

It is well-known that nicotine is subject to oxidation and accordingly, it may be advantageous to incorporate one or more anti-oxidants, such as, e.g., ascorbyl palmitate and/or sodium ascorbate, in a composition according to the invention. The one or more anti-oxidants may be present in a concentration of from about 0.05% to about 0.3% by weight, such as, e.g., from about 0.1% to about 0.25% or from about 0.15% to about 0.2% in the pharmaceutical composition mixture.

Various other substances can be added to the compositions of the present invention. Such components may be provided in a powder or granulated form for mixing with the other components disclosed herein, or otherwise may be provided in liquid form. Most preferably, additional components when provided in a powder or granulated form are employed in the form of parts or pieces that have an average particle size less than about 50 microns. According to some aspects, the average particle size of the components may be about 25 microns or less. The moisture content of the components provided in a powder or granulated form may vary. The particular percentages and choice of ingredients will vary depending upon the desired flavor, texture, and other characteristics.

For example, excipients such as fillers or carriers for active ingredients (e.g., calcium polycarbophil, microcrystalline cellulose, hydroxypropylcellulose, sodium carboxymethylcellulose, cornstarch, silicon dioxide, calcium carbonate, lactose, and starches including potato starch, maize starch, etc.), binders, thickeners, film formers and binders (e.g., hydroxypropyl cellulose, hydroxypropyl methylcellulose, acacia, sodium alginate, xanthan gum and gelatin), antiadherents (e.g., talc), glidants (e.g., colloidal silica), humectants (e.g., glycerin), preservatives and antioxidants (e.g., sodium benzoate and ascorbyl palmitate), flavorants, surfactants (e.g., polysorbate 80), dyes or pigments (e.g., titanium dioxide or D&C Yellow No. 10), and/or lubricants or processing aids (e.g., calcium stearate or magnesium stearate) are added to the compositions in certain embodiments.

Exemplary filler materials include, but are not limited to, grains (including processed grains and puffed grains), maltodextrin, dextrose, calcium carbonate, calcium phosphate, starches (e.g., corn starch), flours (e.g., rice flour), lactose, modified or natural cellulosic materials (e.g., finely divided cellulose, microcrystalline cellulose), bran fibers, vegetable fiber materials such as sugar beet fiber materials (e.g., FIBREX brand filler, available from International Fiber Corporation), and the like. In certain embodiments, the products disclosed herein can comprise a polysaccharide filler and a starch filler. Specific fillers that are advantageously incorporated within the compositions and products of the present disclosure include, but are not limited to, rice flour, maltodextrin, and/or calcium carbonate. In preferred embodiments, one, two, or all three of these fillers are incorporated within a pharmaceutical composition or product as described herein. In some embodiments, the filler material comprises one or more starches and, in certain embodiments, selection of the specific starch or starches can impact the textural properties of the composition and product into which it is incorporated. See, e.g., U.S. Pat. App. Publ. No. 2013/0118512 to Jackson et al., which is incorporated herein by reference.

The total filler content of the compositions of the invention, where present, will typically range from about 5 to about 75 weight percent based on total dry weight of the product, such as about 5 to about 50 weight percent, about 8 to about 50 weight percent, about 5 to about 25 weight percent, about 8 to about 25 weight percent, or about 8 to about 15 weight percent. The total filler content of the product, where present will typically be at least about 5 weight percent, at least about 10 weight percent, or at least about 15 weight percent based on total dry weight of the product. The filler content of the products will typically not exceed about 90 weight percent, such as no more than about 85 weight percent, no more than about 80 weight percent, no more than 75 weight percent, or no more than about 50 weight percent. In other embodiments, the compositions may not comprise a large amount of filler and may, for example, instead comprise a majority (at least 50% by weight) of one or more sugar alcohols as disclosed above. In fact, certain compositions can contain little to none of the fillers disclosed herein.

A binder or binders can, in some embodiments, be incorporated in an amount sufficient to provide the desired physical attributes and physical integrity to the pharmaceutical composition. Binders can be employed, e.g., in an amount of at least about 1% by dry weight, at least about 2% by dry weight, or at least about 5% by dry weight, such as between about between about 5 percent and about 20 percent by dry weight. Binders include, but are not limited to, pre-gelatinized corn starch, pre-gelatinized rice starch, povidone, sodium carboxymethylcellulose (CMC) and other modified cellulosic types of binders, sodium alginate, polydextrose, and starch-based binders. In certain embodiments, the binder material includes a natural gum. As used herein, a natural gum refers to a polysaccharide material of natural origin that is useful as a thickening, binding, or gelling agent. Representative natural gums derived from plants, which are typically water soluble to some degree, include agar agar, xanthan gum, guar gum, gum arabic, ghatti gum, gum tragacanth, karaya gum, locust bean gum, gellan gum, and combinations thereof. When present, natural gum binder materials are typically present in an amount of at least about 1 weight percent or at least about 2 weight percent (e.g., between about 1 and about 10 weight percent, between about 1 and about 5 weight percent or between about 2 and about 5 weight percent).

As referenced above, tobacco-derived, protein-enriched material as disclosed herein can, in some embodiments, provide binding capabilities. As such, it can be used in combination with any one or more of the other types of binders disclosed herein or can replace one or more of the other types of binders disclosed herein, in whole or in part.

A humectant (e.g., glycerin or propylene glycol) may be employed in an amount sufficient to provide desired moisture attributes to the protein-enriched pharmaceutical compositions and products disclosed herein. Further, in some instances, the humectant may impart desirable flow characteristics to the composition. When present, a representative amount of humectant is at least about 0.1 weight percent or at least about 0.2 weight percent, but will typically make up less than about 20 percent of the total weight of the composition (e.g., about 1 weight percent to about 20 weight percent, such as from about 1 weight percent to about 15 weight percent or about 2 weight percent to about 15 weight percent).

A disintegration or compressibility aid can, in some embodiments, be incorporated into the compositions and products disclosed herein. Exemplary disintegration or compressibility aids include microcrystalline cellulose, croscarmellose sodium, crospovidone, calcium carbonate, and sodium starch glycolate. The optional disintegration or compressibility aid can be incorporated in amounts of at least about 0.5 percent by dry weight, at least about 1 percent by dry weight, or at least about 2 percent by dry weight. For example, in certain embodiments, the compositions and products disclosed herein can comprise between about 0.5 and about 10 percent by weight, such as between about 1 and about 5 percent by weight of a disintegration or compressibility aid.

Certain types of nicotine-containing products also can have outer coatings composed of ingredients capable of providing acceptable outer coatings (e.g., an outer coating can be composed of ingredients such as carnauba wax, and pharmaceutically acceptable forms of shellacs, glazing compositions and surface polish agents). Application of a coating can be accomplished using techniques such as airless spraying, fluidized bed coating, use of a coating pan, or the like. Materials for use as a coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Certain representative pharmaceutical compositions may incorporate about 1 to about 10 percent by weight protein-enriched material (e.g., tobacco extract comprising at least about 50%, at least about 60%, at least about 70%, at least about 80%, at least about 90%, at least about 95%, at least about 98%, or at least about 99% RuBisCO and/or F2 protein), about 20 to about 80 percent by weight of one or more sugar alcohols, optionally about 10 to about 50 percent by weight of one or more fillers, and about 0.05 to about 2 percent of one or more nicotinic compounds. Certain specific embodiments further comprise about 2 to about 10 percent by weight humectant.

Formulations of the present invention may include short-term, rapid-onset, rapid-offset, controlled release, sustained release, delayed release, and pulsatile release formulations, providing the formulations achieve administration of a nicotinic compound as described herein. See Remington's Pharmaceutical Sciences (18^{th} ed.; Mack Publishing Company, Eaton, Pa., 1990), which is incorporated herein by reference in its entirety. According to one aspect, a pharmaceutical product as disclosed herein is preferably capable of lasting in the user's mouth for between about 1 and about 30 minutes until it completely dissolves.

For example, solid dosage forms may be formulated so as to provide a delayed release of the active agent (i.e., the nicotinic compound), such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Solid dosage forms according to the present invention may also be sustained release (i.e., releasing the active agent over a prolonged period of time), and may or may not also be delayed release. Sustained release formulations are known in the art and are generally prepared by dispersing an active agent within a matrix of a gradually degradable or hydrolyzable material, such as an insoluble plastic, a hydrophilic polymer, or a fatty compound. Alternatively, a solid dosage form may be coated with such a material.

Compositions can be co-extruded, laminated or formed so as to have sandwich-type forms; and hence the location of nicotine and other ingredients can be controlled in order to provide the desired features such as performance, behavior, interaction or non-interaction with other ingredients, storage stability, and the like. In addition, mixtures of component ingredients can be formulated and manufactured into core/shell types of configurations (i.e., products that have an inner region and at least one additional overlayer), with the various regions of such products having differing overall compositions or properties. Thus, for example, the nicotinic compound can have a relatively high concentration towards the inner region of the product, or a relatively high concentration towards the outer region of the product.

One particularly preferred type of a representative composition incorporating nicotine as an active ingredient, and that comprises nicotine in an orally provided form, has the form of a lozenge, tablet, pellet, microtab, or other tablet-type product. See, for example, the types of nicotine-containing lozenges, lozenge formulations, lozenge formats and configurations, lozenge characteristics and techniques for formulating or manufacturing lozenges set forth in U.S. Pat. No. 4,967,773 to Shaw; U.S. Pat. No. 5,110,605 to Acharya; U.S. Pat. No. 5,733,574 to Dam; U.S. Pat. No. 6,280,761 to Santus; U.S. Pat. No. 6,676,959 to Andersson et al.; U.S. Pat. No. 6,248,760 to Wilhelmsen; and U.S. Pat. No. 7,374,779; US Pat. Pub. Nos. 2001/0016593 to Wilhelmsen; 2004/0101543 to Liu et al.; 2006/0120974 to Mcneight; 2008/0020050 to Chau et al.; 2009/0081291 to Gin et al.; and 2010/0004294 to Axelsson et al.; which are incorporated herein by reference.

The amount of the composition of the invention contained within each piece or unit of lozenge type of product can vary. For example, a representative unit for lozenge products generally weighs at least about 100 mg, often at least about 200 mg, and frequently at least about 300 mg; while the weight of a representative unit for such products generally does not exceed about 1.5 g, often does not exceed about 1 g, and frequently does not exceed about 0.75 g.

The amount of active ingredient within the overall composition can vary. For a composition intended for oral consumption by insertion into the mouth of the subject (e.g., a lozenge or the like), the amount of nicotine within each dosage piece or unit typically is at least about 0.5 mg, generally is at least 1 mg, often is at least about 1.5 mg, and frequently is at least about 2 mg; while the amount of nicotine within each piece typically does not exceed about 10 mg, generally does not exceed about 8 mg, often does not exceed about 6 mg, and frequently does not exceed about 5 mg, calculated as nicotine base. Exemplary types of such products can incorporate about 2 mg, about 2.5 mg, about 3 mg, about 3.5 mg and about 4 mg of nicotine per piece or unit, calculated as nicotine base.

Compositions of the present invention incorporate a pharmaceutically effective amount of nicotine. The dose of active ingredient (i.e., all the various nicotine forms) is preferably that amount effective to treat some symptoms of, or prevent occurrence of the symptoms of, the condition, disease, or disorder from which the subject or patient suffers. By “effective amount,” “therapeutic amount,” or “effective dose” is meant that amount sufficient to elicit the desired pharmacological or therapeutic effects, thus resulting in effective prevention or treatment of the condition, disease, or disorder. Thus, an effective amount of active ingredient is an amount sufficient to enter relevant regions of the body (e.g., to pass across the blood-brain barrier of the subject), to bind to relevant receptor sites in the CNS and PNS of the subject, and/or to elicit neuropharmacological effects (e.g., elicit neurotransmitter secretion, thus resulting in effective prevention or treatment of the condition, disease, or disorder). Prevention of the disorder is manifested, for example, by delaying the onset of the symptoms of the condition, disease, or disorder. Treatment of the disorder is manifested by, for example, a decrease in the symptoms associated with the condition, disease, or disorder or an amelioration of the reoccurrence of the symptoms thereof.

For compositions of the present invention, the intended daily dose of the active ingredient can vary. The overall dose of active ingredient can depend upon factors such as the weight of the subject ingesting the composition, the condition being treated, the state or severity of the disease or disorder being treated, the desired pharmacological effect, or other such factors. Typically, the amount of nicotine active ingredient, calculated as nicotine base, administered to a subject per day is at least about 2 mg, often is at least about 4 mg, and frequently is at least about 10 mg. Typically, the amount of nicotine active ingredient administered to a subject per day does not exceed about 60 mg, often does not exceed about 50 mg, and frequently does not exceed about 40 mg. See also, for example, the types of dosing regimens and administration techniques set forth in U.S. Pat. No. 5,593,684 to Baker et al.; U.S. Pat. No. 6,660,754 to Kyle et al.; and US Pat. Pub. Nos. 2004/0006113 to Sachs; 2005/0214229 to Pinney et al.; 2008/0124283 to Andersen; and 2009/0293895 to Axelsson et al.; which are incorporated herein by reference.

Representative compositions incorporating nicotine as an active ingredient can have various types of formats and configurations, and as a result, the character, nature, behavior, consistency, shape, form, size and weight of the composition can vary. The shape of a representative composition can be generally spherical, cylindrical (e.g., ranging from the general shape of a flattened disc to the general shape of a relatively long, slender stick), helical, obloid, square, rectangular, or the like; or the composition can have the form of a bead, granular powder, crystalline powder, capsule, film, strip, gel, or the like. The shape of the composition can resemble a wide variety of pill, tablet, lozenge, capsule, caplet, pouch and gum types of products that traditionally have been employed for the administration of pharmaceutical types of products. The general nature of a representative composition can be soft or hard to the feel, or of intermediate softness or hardness; and as such, the composition can be considered to be malleable, flexible, chewy, resilient, brittle, or the like. When administered orally, various components of the product can be considered to be readily dispersible or slow to disperse, or those various components can dissolve at varying rates (e.g., from relatively fast to relatively slow). As a result, for compositions ingested by insertion in the mouth of the human subject, the release rate of active ingredient during use of the product can vary from relatively fast to relatively slow, depending upon factors such as the design of the product and the use of product by the subject using that product. See also, by way of example, the types of products proposed in U.S. Pat. No. 4,655,231 to Ray et al.; U.S. Pat. No. 5,147,654 to Place et al.; U.S. Pat. No. 5,543,424 to Carlsson et al.; U.S. Pat. No. 6,268,386 to Thompson; U.S. Pat. No. 6,319,510 to Yates; U.S. Pat. No. 6,488,953 Halliday et al.; U.S. Pat. No. 6,709,671 to Zerbe et al.; U.S. Pat. No. 7,025,983 to Leung et al.; U.S. Pat. No. 7,105,173 to Rolling; U.S. Pat. No. 7,115,297 to Stillman; U.S. Pat. No. 7,435,749 to Knight; and U.S. Pat. No. 7,491,406 to Leung et al.; and US Pat. Pub. Nos. 2006/0198873 to Chan et al.; 2006/0240087 to Houze et al.; 2006/0204559 to Bess et al.; 2007/0269492 to Steen et al.; 2008/0020050 to Chau et al.; 2008/0286340 to Andersson et al.; 2008/0292683 to Sanghvi et al.; and 2009/0004248 to Bunick et al.; which are incorporated herein by reference.

The presence of protein-enriched material (e.g., tobacco-derived protein-enriched material) in a pharmaceutical composition can enhance a pharmaceutical composition in a variety of ways, depending on the nature of the protein-enriched material and the type of composition to which it is added. For example, in some embodiments, protein-enriched materials can serve functional purposes within pharmaceutical compositions, such as binder or filler functions. Certain protein-enriched materials can serve as a replacement for one or more traditional components of a pharmaceutical product.

In certain embodiments, the nicotine-containing pharmaceutical composition is transparent or translucent as defined herein. Transparency/translucency can be determined by any means commonly used in the art; however, it is commonly measured by spectrophotometric light transmission over a range of wavelengths (e.g., from about 400-700 nm). Transmission measurements for the nicotine-containing products of the present invention are typically comparable to or higher than those of traditional nicotine-containing products. Translucency can also be confirmed by visual inspection by simply holding the product up to a light source and determining if light travels through the product in a diffuse manner.

The manners and methods used to formulate and manufacture the nicotine-containing composition can vary. Typical conditions associated with manufacture of pharmaceutical types of products include control of heat and temperature (i.e., the degree of heat to which the various ingredients are exposed during manufacture and the temperature of the manufacturing environment), moisture content (e.g., the degree of moisture present within individual ingredients and within the final composition), humidity within the manufacturing environment, atmospheric control (e.g., nitrogen atmosphere), airflow experienced by the various ingredients during the manufacturing process, and other similar types of factors. Additionally, various process steps involved in product manufacture can involve selection of certain solvents and processing aids, use of heat and radiation, refrigeration and cryogenic conditions, ingredient mixing rates, and the like. The manufacturing conditions also can be controlled due to selection of the form of various ingredients (e.g., solid, liquid, or gas), particle size or crystalline nature of ingredients of solid form, concentration of ingredients in liquid form, or the like. Ingredients can be processed into the desired composition by techniques such as extrusion, compression, spraying, and the like.

In certain embodiments, the products of the invention are prepared by first preparing a dry mixture of ingredients. The composition of the first mixture of ingredients can vary; however, it typically comprises a sugar substitute and may contain various optional additional substances (e.g., fillers, further sweeteners, and/or flavorings). Typically, the first mixture of ingredients does not contain the nicotinic compound. Separately, a wet mixture of ingredients is prepared. The composition of the second mixture can also vary; however, it typically comprises the tobacco-derived protein-enriched material (preferably in hydrated form) and the nicotinic compound. The second mixture can also, in various embodiments, further comprise one or more humectants. The first and second mixtures are them combined. The means by which the components can be combined in this manner can vary. In some embodiments, they may be combined in a mixer. In some embodiments, an agglumerator or granulator is used (wherein wet and dry mixtures can be pre-combined or wherein the wet mixture can be combined with the dry mixture within the agglumerator/granulator).

The combined mixture is then formed into the desired shape. This combined mixture can be formed directly or can be dried (e.g., using heat) prior to being formed. In certain embodiments, the mixture is poured directly into molds, formed (e.g., rolled or pressed) into the desired shape, or extruded. If desired, the mixture can be extruded or injection molded. In certain embodiments, the mixture is formed or extruded into a mold of desired shape in an enclosed system, which may require decreased temperature and which may limit evaporation of certain mixture components. For example, such a system may limit the evaporation of volatile components including, but not limited to, the nicotinic compound and/or flavorants. In other embodiments, the mixture can be pressed or compacted, e.g., following drying, in a compacting machine or pellet press hopper. It is noted that, in certain embodiments, one or more additional ingredients can be added to the mixture prior to the pressing/compacting step. In some embodiments, the mixture or the extrudate can be processed in a spheronizer/marumerizer to provide bead-like pellets.

The preparation can be conducted at atmospheric pressure or under vacuum, but in certain embodiments, at least a portion of the preparation is typically conducted at atmospheric pressure and some of the preparation (e.g., where an agglumerator or granulator is used) can be conducted under vacuum. The temperature at which the disclosed compositions are prepared can also vary. Advantageously, certain compositions can be prepared largely or wholly at ambient temperature. However, in some embodiments, one or more of the components can be heated at various stages of the disclosed processes. In one embodiment, a final composition (e.g., an extruded or molded piece or a granulated material) can be dried at elevated temperature.

Other methods of producing nicotine-containing products are also intended to be encompassed herein. In use, the compositions of the present invention are typically administered in a form adapted for buccal or sublingual delivery. In certain embodiments, the compositions are in a form suitable for oral ingestion. For example, nicotine-containing compositions can be administered and employed using the manners and methods typically used for the administration of traditional types of nicotine-containing products, e.g., lozenges.

The compositions of the present invention can be used for treatment of a wide variety of conditions, diseases, and disorders responsive to stimulation of one or more types of nicotinic acetylcholinergic receptors (nAChRs). The compositions can be used to treat those types of conditions, diseases, and disorders that have been reported to be treatable through the use or administration of nicotine as an agonist of nAChRs. As such, the compositions can be used to treat various CNS conditions, diseases, and disorders, and the compositions also can be used as smoking cessation aids (i.e., as components of NRT). Exemplary conditions, diseases or disorders that can be treated include cognitive disorders such as Alzheimer's disease and attention deficit disorder, schizophrenia, Parkinson's disease, Tourette's syndrome, ulcerative colitis, dry eye disease, hypertension, obesity, and hemorrhoids. Compositions of the invention may also find use as a treatment to reduce stress or pain.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### EXPERIMENTAL

Aspects of the present invention is more fully illustrated by the following examples, which are set forth to illustrate certain aspects of the present invention and is not to be construed as limiting thereof.

### Example 1: NRT Pellets a (RuBisCO as Binder)

### Dry Blend:

| Ingredient | Weight percent | Grams per batch |
|---|---|---|
| Mannitol powder | 37.0 | 369.2 |
| Maltodextrin | 24.0 | 239.5 |
| Calcium carbonate | 20.0 | 199.6 |
| Mint flavor | 4.5 | 44.9 |
| Potassium carbonate | 4.0 | 39.9 |
| Citric acid | 1.0 | 10.0 |
| Sucralose | 0.5 | 5.0 |

### Liquid (Spray Binder):

| | | Grams |
|---|---|---|
| Ingredient | Weight percent | per batch |
| Tobacco-derived RuBisCO | 3.0 | 29.9 |
| Powder | | |
| Glycerin | 5.0 | 49.9 |
| Tobacco extract distillate | 1.0 | 199.6 |
| (4.5% nicotine v/v, | | |
| remainder water) | | |

Tobacco-derived RuBisCO powder is combined with the tobacco extract distillate solution and the mixture is stirred until well hydrated. Glycerin is added to the mixture and stirred thoroughly. The dry blend ingredients are separately combined and the RuBisCO-containing solution is added to the dry blend mixture. The resulting mixture is mixed for 5-10 minutes and transferred to a multigrain extruder. The mixture is extruded through a 2-4 mm die and the extrudate is transferred to a spheronizer/marumerizer, wherein the extrudate is spheronized to provide the product in the form of beads.

### Example 2: NRT Pellets B (RuBisCO as Binder)

### Dry Blend:

| Ingredient | Weight percent | Grams per batch |
|---|---|---|
| Potassium carbonate | 4.0 | 39.9 |
| Sodium chloride | 5.0 | 49.9 |
| Sucralose | 0.5 | 5.0 |
| Citric acid | 1.0 | 10.0 |
| Erythritol | 24.5 | 244.5 |
| Isomalt | 45.0 | 449.1 |
| Mint flavor | 4.5 | 44.9 |

### Liquid (Spray Binder):

| | | Grams |
|---|---|---|
| Ingredient | Weight percent | per batch |
| Tobacco-derived RuBisCO | 2.5 | 25.0 |
| Powder | | |
| Glycerin | 12.5 | 124.7 |
| Nicotine bitartrate dihydrate | 1.0 | 9.98 |
| Water | 298.6 | 0.66 |

Tobacco-derived RuBisCO powder is dispersed in warm water and hydrated overnight. Glycerin and the nicotine bitartrate are combined and mixed thoroughly and then combined with the hydrated RuBisCO. The dry blend ingredients are separately combined and the RuBisCO-containing mixture is added to the dry blend mixture. The resulting mixture is mixed for about 3 minutes and transferred to an extruder. The mixture is extruded through a 2-4 mm die and the extrudate is transferred to a spheronizer/marumerizer, wherein the extrudate is spheronized to provide the product in the form of beads. The beads are dried at 24-45° C. at 25-45% relative humidity for 18-24 hours.

### Example 3: NRT Lozenge (RuBisCO as Binder)

### Dry Blend:

| | | Grams |
|---|---|---|
| Ingredient | Weight percent | per batch |
| Sucralose | 0.5 | 45.4 |
| Calcium carbonate | 5 | 453.60 |
| Isomalt powder | 31 | 2812.3 |
| Erythritol powder | 18 | 1633.0 |
| Microcrystalline cellulose | 3 | 272.2 |
| Potassium carbonate | 5 | 453.6 |
| Pregelatinized rice starch | 7 | 635.0 |

### Liquid Spray (Binder):

| | | Grams |
|---|---|---|
| Ingredient | Weight percent | per batch |
| NaCl | 2.5 | 226.8 |
| Tobacco-derived RuBisCO | 5.5 | 499.0 |
| Powder | | |
| Nicotine bitartrate dihydrate | 1.5 | 136.1 |
| Water | | 7756.56 |

### Final Blend:

| Ingredient | Weight percent | Grams per batch |
|---|---|---|
| Mint flavor powder | 4 | 362.9 |
| Silicon dioxide | 1 | 90.7 |
| Magnesium stearate | 0.5 | 45.4 |
| Stearic acid | 0.5 | 45.4 |

The nicotine bitartrate is dissolved in water and salt is added thereto. The RuBisCO is slowly added and the mixture is stirred until fully hydrated. The dry ingredients are separately combined and mixed for 15 minutes and then transferred to a fluidized bed agglumerator or granulator. The liquid binder spray solution is injected or sprayed onto the dry blend and mixed with the dry blend in the agglumerator/granulator operated under vacuum. The material is heated at 22-65° C. to dry the formed granules and the granules are then mixed with the final blend ingredients in a mixer for another 15 minutes. This formulation is transferred to a compacting machine or pellet press hopper, wherein the powder formulation is pressed into 190-250 mg pellets.

Generally, the inclusion of RuBisCO (e.g., as a binder and/or as a filler) in various types of products provides at least some degree of the desired effects (e.g., binding and/or filling). RuBisCO generally provides properties comparable to traditional binders or fillers, depending how it is incorporated within the product. Accordingly, the tobacco-derived RuBisCO disclosed herein can function as a replacement or substitute for traditional binders or fillers in various pharmaceutical compositions and products.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims.

Although specific terms are employed herein, they are used in a generic and descriptive sense only and not for purposes of limitation.

What is claimed:

1. A protein-enriched pharmaceutical product comprising:
a nicotinic compound;
a protein-enriched, tobacco-derived material in an amount of at least about 2 percent by dry weight; and
one or more sugar alcohols in an amount of at least about 10 percent by dry weight,
wherein the protein-enriched, tobacco-derived material comprises at least about 60 percent tobacco-derived protein by dry weight.

2. The protein-enriched pharmaceutical product of claim 1, wherein the protein-enriched, tobacco-derived material comprises at least about 80 percent tobacco-derived protein by dry weight.

3. The protein-enriched pharmaceutical product of claim 2, wherein at least about 50 percent of the tobacco-derived protein by dry weight is RuBisCO.

4. The protein-enriched pharmaceutical product of claim 2, wherein at least about 80 percent of the tobacco-derived protein by dry weight is RuBisCO.

5. The protein-enriched pharmaceutical product of claim 2, wherein at least about 50 percent of the tobacco-derived protein by dry weight is F2 proteins.

6. The protein-enriched pharmaceutical product of claim 1, wherein at least a portion of the nicotinic compound is in the form of a free base, a salt, a complex, or a solvate.

7. The protein-enriched pharmaceutical product of claim 6, wherein the nicotinic compound is nicotinic polacrilex.

8. The protein-enriched pharmaceutical product of claim 6, wherein the nicotinic compound is sorbed onto a porous particulate carrier.

9. The protein-enriched pharmaceutical product of claim 6, wherein the porous particulate carrier comprises microcrystalline cellulose.

10. The protein-enriched pharmaceutical product of claim 1, wherein the nicotinic compound is present in an amount of about 0.01 to about 2 percent by dry weight.

11. The protein-enriched pharmaceutical product of claim 1, wherein the one or more sugar alcohols are selected from the group consisting of erythritol, arabitol, ribitol, isomalt, maltitol, dulcitol, iditol, mannitol, xylitol, lactitol, sorbitol, and combinations thereof.

12. The protein-enriched pharmaceutical product of claim 1, wherein the one or more sugar alcohols are present in an amount of from about 10 percent to about 80 percent by dry weight.

13. The protein-enriched pharmaceutical product of claim 1, wherein the one or more sugar alcohols are present in an amount of from about 30 percent by weight to about 70 percent by weight.

14. The protein-enriched pharmaceutical product of claim 1, further comprising a binder in an amount of between about 2 percent and about 10 percent by dry weight.

15. The protein-enriched pharmaceutical product of claim 14, wherein the binder comprises pregelatinized rice starch.

16. The protein-enriched pharmaceutical product of claim 1, further comprising one or more fillers in an amount of between about 5 percent and about 50 percent by dry weight.

17. The protein-enriched pharmaceutical product of claim 16, wherein the one or more fillers are selected from the group consisting of maltodextrin, calcium carbonate, and combinations thereof.

18. The protein-enriched pharmaceutical product of claim 1, further comprising an additive selected from the group consisting of flavorants, sweeteners, binders, emulsifiers, disintegration aids, humectants, buffering agents, salts, and mixtures thereof.

19. The protein-enriched pharmaceutical product of claim 1, wherein the composition further comprises glycerin.

20. The protein-enriched pharmaceutical product of claim 1, wherein the composition further comprises one or more sweeteners.

21. The protein-enriched pharmaceutical product of claim 1, comprising:
about 2 percent to about 5 percent by dry weight of the protein-enriched, tobacco-derived material;
about 20 percent to about 50 percent by dry weight of the one or more sugar alcohols;
about 0.01 to about 0.5 percent by dry weight of nicotine;
a filler in an amount of about 30 to about 50 percent by dry weight; and
a humectant in an amount of about 1 to about 10 percent by dry weight.

22. The protein-enriched pharmaceutical product of claim 1, comprising:
about 1 percent to about 5 percent by dry weight of the protein-enriched, tobacco-derived material;
about 0.5 to about 2 percent by weight of a nicotine salt;
about 50 percent to about 75 percent by dry weight of the one or more sugar alcohols; and
a humectant in an amount of about 5 to about 15 percent by dry weight.

23. The protein-enriched pharmaceutical product of claim 1, comprising:
about 2 percent to about 8 percent by dry weight of the protein-enriched, tobacco-derived material;
about 1 to about 2 percent of a nicotine salt;
about 20 percent to about 60 percent by dry weight of the one or more sugar alcohols;
a filler in an amount of about 2 to about 10 percent by weight; and
a binder in an amount of about 2 to about 10 percent by dry weight.

24. A method of preparing a protein-enriched pharmaceutical product, comprising:
combining a dry mixture comprising one or more sugar alcohols in an amount of at least about 10 percent by dry weight with a wet mixture comprising a protein-enriched, tobacco-derived material and a nicotinic compound, wherein the protein-enriched, tobacco-derived material comprises at least about 60 percent tobacco-derived protein by dry weight;
and processing the combined mixture to give a protein-enriched pharmaceutical product, wherein the product comprises at least about 2 percent by dry weight of the protein-enriched, tobacco-derived material.

25. The method of claim 24, wherein the processing comprises extruding.

26. The method of claim 24, wherein the processing comprises compacting.

27. The method of claim 24, wherein the protein-enriched, tobacco-derived material comprises at least about 80 percent tobacco-derived protein by dry weight.

28. The method of claim 24, wherein the protein-enriched pharmaceutical product is in the form of pellets.

29. The method of claim 24, wherein the protein-enriched pharmaceutical product is in the form of a lozenge or tablet.
